(19) United States
(12) Patent Application Publication
(10) Pub. No.: US 2003/0040660 A1
(43) Pub. Date: Feb. 27, 2003

(54) Method for diagnosing and distinguishing traumatic brain injury and diagnostic devices for use therein

(76) Inventors: George Jackowski, Kettleby (CA); Eric B. Stanton, Burlington (CA); Miyoko Takahashi, North York (CA); Michelle Davey, Mississauga (CA)

Correspondence Address: MCHALE & SLAVIN, 4440 PGA BLVD, SUITE 402, PALM BEACH GARDENS, FL, 33410

(21) Appl. No.: 09/940,698

(22) Filed: Aug. 27, 2001

(51) Int. Cl. A61B 5/00

(52) U.S. Cl. 600/300

(57) ABSTRACT

Methods of implementing rapid assessment of patients presenting with brain trauma injury in humans are provided. These methods comprise presenting a subject possibly suffering from traumatic brain injury, selecting one or more markers to detect injury, tracking evolution of injury by tracking marker concentration over time, and determining repetitive injury by the correlation of molecular weight markers appearing over the course of time.

### FIELD OF THE INVENTION

The present invention relates to methods for rapid assessment of subjects suffering from traumatic brain injury. The invention particularly relates to a process and device for one or more markers indicative of cellular damage, and further relates to tracking the concentration of those markers to accurately assess severity of injury. The present invention additionally relates to the diagnosis of repeated injury associated with traumatic brain injury.

### BACKGROUND OF THE INVENTION

Damage to the brain by a physical force is broadly termed traumatic brain injury (TBI) The resulting effect of TBI causes alteration of normal brain processes attributable to changes in brain structure and/or function. There are two basic types of brain injury, open head injury and closed head injury. In an open head injury, an object, such as a bullet, penetrates the skull and damages the brain tissue. Closed head injury is usually caused by a rapid movement of the head during which the brain is whipped back and forth, bouncing off the inside of the skull. Closed head injuries are the most common of the two, which often results from motor vehicle crashes or falls. In a closed head injury, brute force or forceful shaking injures the brain. The stress of this rapid movement pulls apart and stretches nerve fibers or axons, breaking connections between different parts of the brain. In most cases, a resulting blood clot, or hematoma, may push on the brain or around it, raising the pressure inside the head. Both open and closed head injuries can cause severe damage to the brain, resulting in the need for immediate medical attention.

Depending on the type of force that hits the head, varying injuries such as any of the following can result: jarring of the brain within the skull, concussion, skull fracture, contusion, subdural hematoma, or diffuse axonal injury. Though each person's experience is different, there are common problems that many people with TBI face. Possibilities documented include difficulty in concentrating, ineffective problem solving, short and long-term memory problems, and impaired motor or sensory skills; to the point of an inability to perform daily living skills independently such as eating, dressing, or bathing. The most widely accepted concept of brain injury divides the process into primary and secondary events. Primary brain injury is considered to be more or less complete at the time of impact, while secondary injury evolves over a period of hours to days following trauma.

Primary injuries are those commonly associated with emergency situations such as auto accidents, or anything causing temporary loss of consciousness or fracturing of the skull. Contusions, or bruise-like injuries, often occur under the location of a particular impact. The shifting and rotating of the brain inside the skull after a closed brain injury results in shearing injury to the brain's long connecting nerve fibers or axons, which is referred to as diffuse axonal injury. Lacerations are defined as the tearing of frontal and temporal lobes or blood vessels caused by the brain rotating across ridges inside the skull. Hematomas, or blood clots, result when small blood vessels are broken by the injury. They can occur between the skull and the brain (epidural or subdural hematoma), or inside the substance of the brain itself (intracerebral hematoma). In either case, if they are sufficiently large they will compress or shift the brain, damaging sensitive structures within the brain stem. They can also raise the pressure inside the skull and eventually shut off the blood supply to the brain.

Delayed secondary injury at the cellular level has come to be recognized as a major contributor to the ultimate tissue loss that occurs after brain injury. A cascade of physiologic, vascular, and biochemical events is set in motion in injured tissue. This process involves a multitude of systems, including possible changes in neuropeptides, electrolytes such as calcium and magnesium, excitatory amino acids, arachidonic acid metabolites such as the prostaglandins and the leukotrienes, and the formation of oxygen-free radicals. This secondary tissue damage is at the root of most of the severe, long-term deficits a person with brain injury may experience.

Procedures which minimize this damage can be the difference between recovery to a normal or near-normal condition, or permanent disability.

Diffuse blood vessel damage has been increasingly implicated as a major component of brain injury. The vascular response seems to be biphasic. Depending on the severity of the trauma, early changes include an initial rise in blood pressure, an early loss of the automatic regulation of cerebral blood vessels, and a transient breakdown of the blood-brain barrier (BBB). Vascular changes peak at approximately six hours post-injury but can persist for as long as six days. The clinical significance of these blood vessel changes is still unclear, but may relate to delayed brain swelling that is often seen, especially in younger people.

The process by which brain contusions produce brain necrosis is equally complex and is also prolonged over a period of hours. Toxic processes include the release of free oxygen radicals, damage to cell membranes, opening of ion channels to an influx of calcium, release of cytokines, and metabolism of free fatty acids into highly reactive substances that may cause vascular spasm and ischemia. Free radicals are formed at some point in almost every mechanism of secondary injury. Their primary targets are the fatty acids of the cell membrane. A process known as lipid peroxidation damages neuronal, glial, and vascular cell membranes in a geometrically progressing fashion. If unchecked, lipid peroxidation spreads over the surface of the cell membrane and eventually leads to cell death. Thus, free radicals damage endothelial cells, disrupt the BBB, and directly injure brain cells, causing edema and structural changes in neurons and glia. Disruption of the BBB is responsible for brain edema and exposure of brain cells to damaging blood-borne products.

Secondary systemic insults (outside the brain) may consequently lead to further damage to the brain. This is extremely common after brain injuries of all grades of severity, particularly if they are associated with multiple injuries. Thus, people with brain injury may experience combinations of low blood oxygen, blood pressure, heart and lung changes, fever, blood coagulation disorders, and other adverse changes at recurrent intervals in the days following brain injury. These occur at a time when the normal regulatory mechanism, by which the cerebrovascular vessels can relax to maintain an adequate supply of oxygen and blood during such adverse events, is impaired as a result of the original trauma.

The protocols for immediate assessment are limited in their efficiency and reliability and are often invasive.

Immediate treatment for TBI typically involves surgery to control bleeding in and around the brain, monitoring and controlling intracranial pressure, insuring adequate blood flow to the brain, and treating the body for other injuries and infection. Those with mild brain injuries often experience subtle symptoms and may defer treatment for days or even weeks. Once a patient chooses to seek medical attention, observation, neurological testing, magnetic resonance imaging (MRI), positron emission tomography (PET) scan, single-photon emission CT (SPECT) scan, monitoring the level of a neurotransmitter in spinal fluid, computed tomography (CT) scans, and X-rays may be used to determine the extent of the patient's injury. The type and severity of the injury determine further care.

Unfortunately, mild brain injuries often result in long-term disabilities.

According to the Center for Disease Control, national data estimates for 1995-1996 for incidence of traumatic brain injury include the treatment and release of one million patients from hospital emergency departments, wherein for every 230,000 hospitalized who survive, 50,000 die. It is now estimated that every 15 seconds another person in the United States sustains a brain injury and that at least 5.3 million Americans are currently living with a TBI-related disability.

The cost of TBI in the United States regarding such areas as disability, lost work wages, and rehabilitation for resulting various cognitive and movement impairments total approximately 48 billion dollars, with hospitalization costs reaching 32 billion each year. This obviously does not include the human costs, or burdens borne, by those who are injured and their families.

Diagnostic techniques for the early diagnosis of traumatic brain injury and identification of the type of event of TBI are needed to allow a physician to prescribe the appropriate therapeutic drugs at an early stage in the cerebral event.

Various markers for brain injury are proposed and analytical techniques for the determination of such markers have been described in the art. As used herein, the term “marker” refers to a protein or other molecule that is released from the brain during a cerebral event. Such markers include isoforms of proteins that are unique to the brain.

It has been reported in the literature that various biochemical markers have correlated with cerebral events such as traumatic brain injury. Myelin basic protein (MBP) concentration in cerebrospinal fluid (CSF) increases following sufficient damage to neuronal tissue, head trauma, or AIDS dementia. Further, it has been reported that ultrastructural immunocytochemistry studies using anti-MBP antibodies have shown that MBP is localized exclusively in the myelin sheath. S-100 protein is another marker which may be useful for assessing neurologic damage, for determining the extent of brain damage, and for determining the extent of brain lesions.

Thus, S-100 protein has been suggested for use as an aid in the diagnosis and assessment of brain lesions and neurological damage due to brain injury, as in stroke. Neuron specific enolase (NSE) also has been suggested as a useful marker of neurologic damage in the study of brain injury, as in stroke, with particular application in the assessment of treatment.

### PRIOR ART

Herrmann et al. (J. Neurotrauma (2000) 17, 2, 113-133) aim their investigation on the release of neuronal markers, neuron specific enolase (NSE), and S-100B, and their association with intracranial pathologic changes as demonstrated in computerized tomography (CT). Their findings suggest release patterns of S-100B and NSE differ in patients with primary cortical contusions, diffuse axonal injury, and signs of cerebral edema without focal mass lesions. It is also suggested that all serum concentrations of NSE and S-100B significantly correlate with the volume of contusions. Herrmann et al. therefore suggest NSE and S-100B may mirror different pathophysiological consequences of TBI. In a later study, Herrmann et al. (J. Neurol. Neurosurg. Psych. (2001) 70, 1, 95-100) examine the release patterns of neurobiochemical markers of brain damage (NSE and protein S-100) in patients with traumatic brain injury and their predictive value with respect to the short and long term neuropsychological outcome. Serial NSE and S-100B concentrations are analyzed in blood samples taken at the first, second, and third day after traumatic brain injury.

Patients with short and long-term neuropsychological disorders are found to have significantly higher NSE and S-100B serum concentrations and a significantly longer lasting release of both markers. A comparative analysis of the predictive value of clinical, neuroradiological, and biochemical data shows initial S-100B values above 140 ng/L to have the highest predictive power. Therefore, it is suggested the analysis of post-traumatic release patterns of neurobiochemical markers of brain damage might help to identify patients with traumatic brain injury who run a risk of long-term neuropsychological dysfunction.

Raabe et al. (Acta Neurochir. (Wien) (1998) 140, 8, 787-792) investigate the association between the initial levels of serum S-100B protein and NSE and the severity of radiologically visible brain damage and outcome after severe head injury. They suggest there exists a significant correlation between different grades of diffuse injury determined by Marshall classification and initial serum S-100B protein, and between the volume of contusion visible on CT scans and serum S-100B. Further, they suggest serum S-100B may provide superior information about the severity of primary brain damage after head injury.

Raabe and Seifert (Neurosurg. Rev. (2000), 23, 3, 136-138) teach the use of S-100 B protein independently as a serum marker of brain cell damage after severe head injury. Minor head injury is usually defined as a clinical state involving a Glasgow Coma Scale (GCS) score of 13-15; the lower the score the more severe the injury. Patients with severe head injury (GCS≦8) are thought to be the best candidates for this study. Venous blood samples for S-100B protein are taken after admission and every 24 hours for a maximum of 10 consecutive days. Outcome is assessed at 6 months using the Glasgow Outcome Scale. Their findings indicate levels of S-100B are significantly higher in patients with unfavorable outcome compared to those with favorable outcome. In patients with favorable outcome, slightly increased initial levels of S-100B return to normal within 3 to 4 days. However, in patients with unfavorable outcome, initial levels are markedly increased, with a tendency to decrease from day 1 to day 6. After day 6, there tends to be a secondary increase in serum S-100B, indicating secondary brain cell damage. Their preliminary results suggest that serum S-100B protein may be a promising biochemical marker which may provide additional information on the extent of primary injury to the brain and the prediction of outcome after severe head injury. Rothoerl et al. (J. Trauma (1998), 45, 4, 765-767) demonstrate the difference in S-100B serum levels following minor and major head injury. In minor injury, the mean serum level of S-100B within 6 hours of injury is 0.35 μg/L. In major injury with a favorable outcome, the mean serum concentration is shown as 1.2 μg/L, whereas with an unfavorable outcome the mean is 4.9 μg/L. Rothoerl et al. only identify there is a difference, but do not utilize the varying levels in the diagnosis of patients presenting with head trauma. Follow-up on the progress of patient outcome once the patient is discharged is not discussed.

Ingebrigtsen et al. (Neurosurg. (1999), 45, 3, 468-476) are interested in the relation of serum S-100 protein measurements to MRI and neurobehavioral outcome in damage due to minor head injury. Minor head injury in this study consist of patients with a GCS score of 13-15 in whom brain CT scans revealed no abnormalities. Serum levels are initially taken upon hospital admittance and hourly thereafter for 12 hours following injury. Analysis is by a two-site immunoradiometric assay kit. Their findings indicate a mean peak serum level of S-100 to be 0.4 μg/L in 28% of patients which were highest upon initial analysis and would decline thereafter. The patients with MRI revealing contusions also tend to have significantly higher serum S-100 levels. In addition, these patients form a trend toward impaired neuropsychological functioning on measures of attention, memory, and information processing speed, for which all patients are tested at 3 months post-injury. They conclude that measurements of S-100 recently following head injury provide information on the extent of TBI, but most importantly also contribute early prognostic information for identification of patients on later neurobehavioral outcome, specifically prolonged neurobehavioral dysfunction.

Fridriksson et al., (Acad. Emerg. Med. (2000) 7,7,816-820) based on their findings, suggest serum neuron specific enolase as a reliable marker in the prediction of intracranial lesions in children with head trauma. Their studies are based on the findings of Skogseld et al. (Acta NeuroChir. (Wien) (1992), 115, 106-111) and Yamazaki et al. (Surg. Neurol. (1995), 43, 3, 267-271) who suggest that serum NSE levels in patients with head trauma usually peak early afer injury, reflecting the mechanical disruption of brain tissue, and then gradually fall. Although thought to be a reliable marker for predicting intracranial lesions in children, their results indicate elevated serum NSE levels in the acute phase after blunt trauma are neither sensitive nor specific in detecting all lesions. Nearly 25% of patients with intracranial lesions are missed, including patients in dire need of surgical procedure.

Yamazaki et al. (Surg. Neurol. (1995), 43, 3, 267-271) illustrates the diagnostic significance of patients with acute head injury between those who survive and those who die. Blood samples are taken following injury at a mean of 4.3 hours. Serum levels of NSE and MBP are both significantly elevated in the patients who die versus the patients who survive. For NSE, the levels are approximately 51 ng/mL versus 18 ng/mL, respectively. For MBP, the levels are approximately 11 ng/mL versus 1 ng/mL, respectively. This assay of NSE and MBP levels is suggested to provide early prediction of the prognosis in patients with acute head injury.

Myelin basic protein (MBP) is generally thought to be associated with autoimmune disease. However, MBP has also been linked with head trauma. Most significant is the study by Mao et al. (Hua Xi Yi Ke Da Xue Xue Bao (article in Chinese) (1995), 26, 2, 135-137). Serum levels of MBP analyzed by enzyme-linked immunosorbent assay (ELISA) following acute closed head injury appear to show distinctions between type of injury. At a significantly high level of serum MBP (p<0.05) are patients with severe injury such as cerebral contusion or intracerebral hematoma, with no significant difference between them. Much lower are patients with extradural hematoma.

Patients with cerebral concussion show no significant change in serum MBP. Thomas et al. (Lancet (1978), 1, 8056, 113-115) goes one step further to show mean concentrations of MBP in patients with severe intracerebral damage, with or without extracerebral hematoma, at a significantly raised level for two weeks after injury.

U.S. Pat. No. 5,486,204 issued to Clifton teaches a method of treating severe, closed head injury with hypothermia. This is done in order to diminish brain tissue loss when administered during and after ischemia. Such a method includes the administration of medications to control both the effects of the brain injury and to balance the potential deleterious effects to the body of being subjected to reduced temperatures for an extended period. According to the claims, a patient must be cooled for 48 hours. Not only does this method absolutely require a long period of time and proper space to perform this task, but also involves medications to combat the side effects of hypothermia, in addition to those for treating the brain injury.

Methods of assessing and treating head injuries often suggest the administration of pharmaceutical drugs as a blind test to determine the extent of damage. This may not only be costly but also dangerous to a patient on other medications. U.S. Pat. Nos. 6,096,739, 6,090,775, and 5,527,822 all teach a method of treatment involving the administration of a pharmaceutical. U.S. Pat. No. 6,096,739 issued to Feuerstein, uses cytokine inhibitors, or 1,4,5-substituted imidazole compounds and compositions, to treat CNS injuries to the brain. U.S. Pat. No. 6,090,775 issued to Rothwell, et al., uses a compound which treats the conditions of neurological degeneration by interfering with the action of interleukin-1, an agent which affects a wide variety of cells and tissues, directly modifying glial and neuronal function, and is critical in mediating inflammatory conditions. U.S. Pat. No. 5,527,822 issued to Scheiner, describes a method of treatment of traumatic brain injury by administering a butyrolactone derivative. This patent does describe a form of treatment based on a diagnosis of traumatic brain injury based on the presence of intracranial hypertension with direct effects on cerebral perfusion following TBI and leading to acute inflammation.

U.S. Pat. No. 6,052,619 describes the use of a portable electroencephalograph (EEG) instrument to detect and amplify brain waves and convert them into digital data for analysis by comparison with data from normal groups. This is suggested for use in emergencies and brain assessments in a physician's office. Although very useful, the described invention is a medical system to transmit data, not a biochemical testing procedure.

U.S. Pat. No. 6,235,489, to Jackowski, entitled “Method for Diagnosing and Distinguishing Stroke and Diagnostic Devices for Use Therein” is drawn to a method for determining whether a subject has had a stroke and, if so, the type of stroke which includes analyzing the subject's body fluid for at least four selected markers of stroke, namely, myelin basic protein, S100 protein, neuronal specific enolase and a brain endothelial membrane protein such as thrombomodulin or a similar molecule. The data obtained from the analyses provide information as to the type of stroke, the onset of occurrence and the extent of brain damage and allow a physician to determine quickly the type of treatment required by the subject.

What is lacking in the art is a non-invasive point-of-care methodology useful for recent TBI sufferers to enable appropriate measures to be taken for treatment, for example, on-site in emergency situations or over a prolonged period for chronic conditions. Providing a rapid point-of-care test would enable the practitioner to quickly and definitively determine the presence of head trauma. For example, this type of test could be performed by an EMT or upon arrival in the ER. The importance of such a tool can be illustrated by the example of child abuse cases where the infant (shaken baby syndrome) or child may not be able to express what has occurred. The proper authorities could perform the simple, inexpensive test to ensure whether abusive events have occurred and whether these events have been ongoing. In addition, the safety of the infant could be conveniently followed by intermittent testing for further signs of abuse. Another useful example lies in the sports arena. Hockey players and boxers are routinely exposed to constant forces against the head. A simple diagnostic test could determine the immediate effects of an individual concussion, or the build up of repetitive injury with each ensuing match. An acceptable level could be implemented to protect players from dangerous levels of exposure, thus avoiding the devastating effects of secondary injuries. Such techniques can provide data which will allow a physician to rapidly determine the appropriate treatment required by the patient and thereby permit early intervention.

### SUMMARY OF THE INVENTION

The present invention provides a diagnostic test kit and a method for its use that is capable of determining whether a patient has suffered traumatic brain injury and, if so, whether the event is exemplary of primary or secondary conditions. According to the method, a body fluid of the patient is analyzed for at least one molecule which is cell type specific, namely, S-100B, neuron specific enolase (NSE), and myelin basic protein (MBP). The method analyzes the isoforms of the marker proteins which are specific to the brain. The biochemical markers may be utilized singly or in various combinations conclusive of various types of trauma. The analyses of these markers may be carried out on the same sample of body fluid or on multiple samples of body fluid. Different body fluid samples may be taken at the same time or at different time periods.

The information which is obtained according to the method of the invention can be vital to the physician by assisting in the determination of how to treat a patient presenting with symptoms of TBI or suspected of TBI. The data may rule TBI in or out, and differentiate between primary and secondary TBI. The data may also determine whether there is evidence of ongoing or repetitive injury. Further, the method can provide, at an early stage, prognostic information relating to the outcome of intervention which can improve patient selection for appropriate therapeutics and intervention. The method of the invention is diagnostic well before the imaging technologies. By measuring the markers in samples of body fluid taken at different points in time, the progress of the TBI can be ascertained.

The present invention relates to the rapid assessment of a patient presenting with traumatic brain injury. A test involving biochemical markers of neuronal damage is utilized to quantify whether an injury is related to traumatic brain injury. The term “quantify” is used herein to determine the occurrence, to distinguish type, to measure severity, or to conclusively track progression and/or evidence of ongoing or repetitive injury. In addition, the present invention relates to the usefulness of continued monitoring of TBI patients for a period of time. This type of assessment could be very useful in the proper treatment of persons suffering from traumatic brain injury.

Accordingly, it is an objective of the instant invention to provide a method for rapidly diagnosing and distinguishing traumatic brain injury.

It is a further objective of the instant invention to provide such a method which includes analyzing the body fluid of a patient for at least one marker indicative of traumatic brain injury.

It is yet another objective of the instant invention to provide a method which can provide information relating to whether the traumatic brain injury is a result of repetitive injury.

It is a still further objective of the invention to provide diagnostic assay devices for use in the method.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 illustrates a Table of Data relating to Trauma Patients.

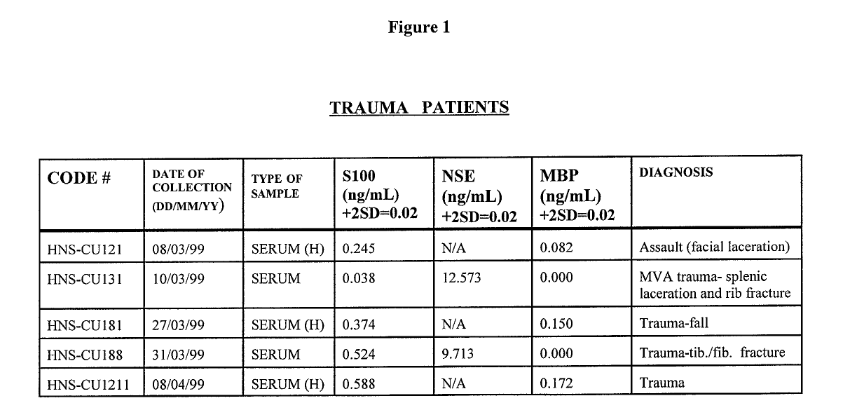

### DETAILED DESCRIPTION OF THE INVENTION

The markers which are analyzed according to the method of the invention are released into the circulation and are present in the blood and other body fluids. Preferably blood, or any blood product that contains them such as, for example, plasma, serum, cytolyzed blood (e.g., by treatment with hypotonic buffer or detergents), and dilutions and preparations thereof is analyzed according to the invention. In another preferred embodiment the concentration of the markers in CSF is measured. Kits for diagnosing traumatic brain injury are also described.

The terms “above normal” and “above threshold” are used herein to refer to a level of a marker that is greater than the level of the marker observed in normal individuals, that is, individuals who are not undergoing a cerebral event, i.e. an injury to the brain which may be ischemic, mechanical or infectious. For some markers, no or infinitesimally low levels of the marker may be present normally in an individual's blood. For others of the markers analyzed for according to the invention, detectable levels may be present normally in blood. Thus, these terms contemplate a level that is significantly above the normal level found in individuals. The term “significantly” refers to statistical significance and generally means a two standard deviation (SD) above normal, or higher, concentration of the marker is present. The assay method by which the analysis for any particular marker protein is carried out must be sufficiently sensitive to be able to detect the level of the marker which is present over the concentration range of interest and also must be highly specific.

The primary markers which are measured according to the present method are proteins which are released by the specific brain cells as the cells become damaged during a cerebral event. These proteins can be either in their native form or immunologically detectable fragments of the proteins resulting, for example, by enzyme activity from proteolytic breakdown. The specific primary markers when mentioned in the present application, including the claims hereof, are intended to include fragments of the proteins which can be immunologically detected. By “immunologically detectable” is meant that the protein fragments contain an epitope which is specifically recognized by a cognate antibody.

As mentioned previously, the markers analyzed according to the method of the invention are cell type specific. Myelin basic protein (MBP) is a highly basic protein, localized in the myelin sheath, and accounts for about 30% of the total protein of the myelin in the human brain. The protein exists as a single polypeptide chain of 170 amino acid residues which has a rod-like structure with dimensions of 1.5×150 nm and a molecular weight of about 18,500 Dalton. It is a flexible protein which exists in a random coil devoid of a helices β conformations.

The increase of MBP concentration in blood and CSF in cerebral hemorrhage is highest almost immediately after the onset. A normal value for a person who has not had a cerebral event is from 0.00 to about 0.016 ng/mL. MBP has a half-life in serum of about one hour and is a sensitive marker for cerebral hemorrhage.

The S-100 protein is a cytoplasmic acidic calcium binding protein found predominantly in the gray matter of the brain, primarily in glia and Schwann cells. The protein exists in several homo- or heterodimeric isoforms consisting of two immunologically distinct subunits, alpha (MW=10,400 Dalton) and beta (MW=10,500 Dalton) while the S-100ασ is the homodimer au which is found mainly in striated muscle, heart and kidney. The S-100B isoform is the 21,000 Dalton homodimer ββ. It is present in high concentration in glial cells and Schwann cells and is thus tissue specific. It is released during acute damage to the central nervous system and is a sensitive marker for cerebral infarction. The S-100B isoform is a specific brain marker released during acute damage to the central nervous system. It is eliminated by the kidney and has a half-life of about two hours in human serum. Repeated measurements of S-100 serum levels are useful to follow the course of neurologic damage.

The enzyme, enolase (EC 4.2. 1.11) catalyzes the interconversion of 2-phosphoglycerate and phosphoenolpyruvate in the glycolytic pathway. The enzyme exists in three isoproteins each the product of a separate gene. The gene loci have been designated ENO1, ENO2 and ENO3. The gene product of ENO1 is the non-neuronal enolase (NNE or α), which is widely distributed in various mammalian tissues. The gene product of ENO2 is the muscle specific enolase (MSE or β) which is localized mainly in the cardiac and striated muscle, while the product of the ENO3 gene is the neuron specific enolase (NSE or γ) which is largely found in the neurons and neuroendocrine cells. The native enzymes are found as homo- or heterodimeric isoforms composed of three immunologically distinct subunits, α, β, and γ. Each subunit has a molecular weight of approximately 39,000 Dalton.

The a αγ and γγ enolase isoforms, which have been designated neuron specific enolase (NSE) each have a molecular weight of approximately 80,000 Dalton. It has been shown that NSE concentration in CSF increases after experimental focal ischemia and the release of NSE from damaged cerebral tissue into the CSF reflects the development and size of the infarcts. NSE has a serum half-life of about 48 hours and its peak concentration has been shown to occur later after cerebral artery (MCA) occlusion. NSE levels in CSF have been found to be elevated in acute and/or extensive disorders including subarachnoid hemorrhage and acute cerebral infarction.

The data obtained according to the method indicate whether a traumatic brain injury has occurred and, if so, the type of injury, primary or secondary. Where all markers analyzed are negative, i.e., within the normal range, there is no indication of traumatic brain injury. When the level of any marker analyzed is at least 2SD above the normal range, there is indication of trauma. Depending on which markers and the degree of marker level, severity can be determined. Prior art data have indicated that possible conclusions to be drawn are very high MBP and S-100 are indicative of contusion or intracerebral hematoma; high S-100 but normal after 3-4 days indicates a favorable outcome; high S-100 for 1-6 days and then up again, indicates an unfavorable outcome; high MPB for 2 weeks indicates an unfavorable outcome; raised S-100 with no raise in MBP is indicative of a concussion.

According to another preferred embodiment, a fourth marker, which is from the group of axonal, glial, and neuronal markers analyzed according to the method of the invention, is measured to provide information related to the time of onset of the traumatic brain injury. It should be recognized that the onset of TBI symptoms is not always known, particularly if the patient is unconscious or elderly and a reliable clinical history is not always available. An indication of the time of onset of the TBI can be obtained by relying on the differing release kinetics of brain markers having different molecular weights. The time release of brain markers into the circulation following brain injury is dependent on the size of the marker, with smaller markers tending to be released earlier in the event while larger markers tend to be released later. Thus, in a particularly preferred embodiment, the method and kit for its performance include a fourth antibody which is specific for a fourth marker protein, wherein said fourth marker protein is cell type specific with respect to one of said first, second or third markers and has a correspondingly higher molecular weight than said first, second or third marker, and a fourth labeled antibody which binds to said fourth marker protein.

As stated previously, the level of each of the specific markers in the patient's body fluid can be measured from one single sample or one or more individual markers can be measured in one sample and at least one marker measured in one or more additional samples. By “sample” is meant a volume of body fluid such as blood or CSF which is obtained at one point in time. Further, all the markers can be measured with one assay device or by using a separate assay device for each marker in which case aliquots of the same fluid sample can be used or different fluid samples can be used. It is apparent that the analyses should be carried out within some short time frame after the sample is taken, e.g., within about one-half hour, so the data can be used to prescribe treatment as quickly as possible. It is preferred to measure each of the markers in the same single sample, irrespective of whether the analyses are carried out in a single analytical device or in separate such devices so the level of each marker simultaneously present in a single sample can be used to provide meaningful data.

Generally speaking, the presence of each marker is determined using antibodies specific for each of the markers and detecting immunospecific binding of each antibody to its respective cognate marker. Any suitable immunoassay method may be utilized, including those which are commercially available, to determine the level of each of the specific markers measured according to the invention. Extensive discussion of the known immunoassay techniques is not required here since these art known to those of skill in the art. Typical suitable immunoassay techniques include sandwich enzyme-linked immunoassays (ELISA), radio immunoassays (RIA), competitive binding assays, homogeneous assays, heterogeneous assays, etc. Various of the known immunoassay methods are reviewed in Methods in Enzymology, 70, pp.30-70 and 166-198 (1980). Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include: radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall, Enzyme Immunoassay ELISA and EMIT, Methods of Enzymology, 70,419439 (1980).

A preferred immunoassay method for use according to the invention is a double antibody technique for measuring the level of the marker proteins in the patient's body fluid. According to this method one of the antibodies is a “capture” antibody and the other is a “detector” antibody. The capture antibody is immobilized on a solid support which may be any of various types which are known in the art such as, for example, microtiter plate wells, beads, tubes and porous materials such as nylon, glass fibers and other polymeric materials. In this method, a solid support, e.g., microtiter plate wells, coated with a capture antibody, preferably monoclonal, raised against the particular marker protein of interest, constitutes the solid phase. Diluted patient body fluid, e.g., serum or plasma, typically about 25 μl, standards and controls are added to separate solid supports and incubated. When the marker protein is present in the body fluid it is captured by the immobilized antibody which is specific for the protein. After incubation and washing, an anti-marker protein detector antibody, e.g., a polyclonal rabbit anti-marker protein antibody, is added to the solid support. The detector antibody binds to marker protein bound to the capture antibody to form a sandwich structure. After incubation and washing an anti-IgG antibody, e.g., a polyclonal goat anti-rabbit IgG antibody labeled with an enzyme such as horseradish peroxidase (HRP) is added to the solid support. After incubation and washing, a substrate for the enzyme is added to the solid support followed by incubation and the addition of an acid solution to stop the enzymatic reaction. The degree of enzymatic activity of immobilized enzyme is determined by measuring the optical density of the oxidized enzymatic product on the solid support at the appropriate wavelength, e.g., 450 nm for HRP. The absorbance at the wavelength is proportional to the amount of marker protein in the fluid sample. A set of marker protein standards is used to prepare a standard curve of absorbance vs. marker protein concentration. This method is preferred since test results can be provided in 45 to 50 minutes and the method is both sensitive over the concentration range of interest for each marker and is highly specific.

The assay methods used to measure the marker proteins should exhibit sufficient sensitivity to be able to measure each protein over a concentration range from normal values found in healthy persons to elevated levels, ie., 2SD above normal and beyond. Of course, a normal value range of the marker proteins can be found by analyzing the body fluid of healthy persons. For the S-100B isoform where +2SD=0.02 ng/mL the upper limit of the assay range is preferably about 5.0 ng/mL. For NSE where +2SD=9.9 ng/mL the upper limit of the range is preferably about 60 ng/mL. For MBP, which has an elevated level cutoff value of 0.02 ng/mL, the upper limit of the assay range is preferably about 5.0 ng/mL.

The assays can be carried out in various assay device formats including those described in U.S. Pat. Nos. 4,906,439; 5,051,237 and 5,147,609 to PB Diagnostic Systems, Inc.

The assay devices used according to the invention can be arranged to provide a semi-quantitative or a quantitative result. By the term “semi-quantitative” is meant the ability to discriminate between a level which is above the elevated marker protein value, and a level which is not above that threshold.

As used herein, the term “marker” refers to a protein or other molecule that is released upon trauma to the brain. Such markers include, but are not limited to, proteins or isoforms of such proteins that are unique to the brain, and/or proteins or isoforms thereof that are found in tissues other than the brain.

The assays may be carried out in various formats including, as discussed previously, a microtiter plate format which is preferred for carrying out the assays in a batch mode. The assays may also be carried out in automated immunoassay analyzers which are well known in the art and which can carry out assays on a number of different samples. These automated analyzers include continuous/random access types. Examples of such systems are described in U.S. Pat. Nos. 5,207,987 and 5,518,688 to PB Diagnostic Systems, Inc. Various automated analyzers that are commercially available include the OPUS® and OPUS MAGNUM® analyzers. Another assay format which can be used according to the invention is a rapid manual test which can be administered at the point-of-care at any location. Typically, such point-of-care assay devices will provide a result which is above or below a threshold value, i.e., a semi-quantitative result as described previously.

It should be recognized also that the assay devices used according to the invention can be provided to carry out one single assay for a particular marker protein or to carry out a plurality of assays, from a single volume of body fluid, for a corresponding number of different marker proteins. A preferred assay device of the latter type is one which can provide a semi-quantitative result for the primary marker proteins measured according to the invention, i.e., S-100B, NSE, and MBP. These devices typically are adapted to provide a distinct visually detectable colored band at the location where the capture antibody for the particular marker protein is located when the concentration of the marker protein is above the threshold level. For a detailed discussion of assay types which can be utilized according to the invention as well as various assay formats and automated analyzer apparatus see U.S. Pat. No. 5,747,274 to Jackowski.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A diagnostic method for quantifying a subject suffering from a symptom caused by traumatic brain injury or characteristic of traumatic brain injury, comprising:
a. obtaining a sample of body fluid from a subject;
b. selecting at least one marker appropriate to the condition of said subject suffering from a symptom caused by TBI or characteristic of TBI;
c. measuring concentration of said at least one marker in said sample; and
d. if required, further monitoring said subject as in preceding steps (a), (b), and (c), respectively, until said subject can be fully diagnosed.

2. A method as in claim 1, wherein said sample of body fluid is serum or plasma.

3. A method as in claim 1, wherein said at least one marker is selected from the group consisting of S-100B, neuron specific enolase, and myelin basic protein.

4. A method as in claim 1, wherein said at least one marker is S-100B.

5. A method as in claim 1, wherein said at least one marker is neuron specific enolase.

6. A method as in claim 1, wherein said at least once marker is myelin basic protein.

7. A method as in claim 1, wherein said at least one marker is selected from the group consisting of glial, neuronal, and axonal markers.

8. A method as in claim 1, wherein said measuring concentration is by an immunoassay method.

9. A method as defined in claim 1, wherein each of said analyses is carried out on the same sample of body fluid.

10. A method as defined in claim 1, wherein at least one of said analyses is carried out on a first sample of body fluid and at least another of said analyses is carried out on a second sample of body fluid.

11. A method as defined in claim 10, wherein said first and said second samples of body fluid are taken at different time periods.

12. A method as in claim 1, further including the step of:
tracking concentration of said at least one marker in said subject over a period of time.

13. A method as in claim 12, wherein tracking concentration of said at least one marker is performed by a diagnostic procedure selected from the group consisting of radioimmunoassay and enzyme-linked immunoassay method.

14. A method as in claim 13, wherein each of said immunoassay method comprises contacting said sample of body fluid with an antibody which is specific for said at least one marker.

15. A diagnostic kit for quantifying traumatic brain injury comprising at least three antibodies which are specific for each of three different marker proteins, said antibodies capable of being immobilized on a solid support, wherein:
a. a first marker protein is the beta isoform of S-100 protein and a first antibody is specific therefor,
b. a second marker protein is neuron specific enolase and a second antibody is specific therefor,
c. a third marker protein is myelin basic protein and a third antibody is specific therefor, and
at least three labeled antibodies, each of said labeled antibodies having an affinity for one of said marker proteins.

16. A diagnostic kit as defined in claim 15, wherein each of said three antibodies is immobilized on the same solid support.

17. A diagnostic kit as defined in claim 15, wherein each of said three antibodies is immobilized on a separate solid support.

18. A diagnostic kit as defined in claim 15, wherein at least one of said labeled antibodies comprises an enzyme-labeled antibody.

19. A diagnostic kit as defined in claim 15 and further including a fourth antibody which is specific for a fourth marker protein, wherein said fourth marker protein is a glial, axonal or neuronal cell type having a higher molecular weight than the beta isoform of S-100 or neuronal-specific enolase, respectively, and a fourth labeled antibody which binds to said fourth marker protein.

20. A diagnostic kit as defined in claim 15, wherein said fourth labeled antibody comprises an enzyme-labeled antibody.

21. A method for confirming the occurrence of a traumatic brain injury event comprising:
a. analyzing a body fluid of a patient to detect the presence and concentration of at least one of three markers of traumatic brain injury wherein;
i. a first marker is myelin basic protein,
ii. a second marker is the beta isoform of S100 protein, and
iii. a third marker is neuronal specific enolase, and
b. comparing any of said markers whose presence is detected to specific threshold values of each of the markers to determine the presence of statistically significant concentrations thereof of at least about two standard deviations above normal levels;
wherein said step of comparing at least one of said three markers confirms the occurrence of a traumatic brain injury event.

22. A method as defined in claim 21 wherein said body fluid is selected from the group consisting of blood, blood components and cerebrospinal fluid.

23. A method as defined in claim 21 wherein each of said analyses is carried out on a single sample of body fluid.

24. A method as defined in claim 21 wherein at least one of said analyses is carried out on a first sample of body fluid and at least another of said analyses is carried out on a second sample of body fluid.

25. A method as defined in claim 24 wherein said first and said second samples of body fluid are taken at different time periods.

26. A method as defined in claim 21 wherein at least one of said analyses comprises contacting said body fluid with an antibody which is specific for said marker.

27. A method as defined in claim 26 wherein at least one of said analyses is carried out with an enzyme-labeled immunoassay method.

28. A method as defined in claim 21 and further including the step of analyzing said body fluid for a fourth marker protein, wherein said fourth marker protein is cell type specific with respect to one of said first, second or third markers and has a correspondingly higher molecular weight than said first, second or third marker.

29. A method as defined in claim 28 wherein at least one of said analyses
comprises contacting said body fluid with an antibody which is specific for said marker.

30. A method as defined in claim 28 wherein at least one of said analyses is carried out with an enzyme-labeled immunoassay method.

31. A method as defined in claim 21 and further including the step of analyzing a second sample of a body fluid from said patient for at least one of said three markers, said second sample of body fluid being taken at a time subsequent to the time at which said body fluid analyzed in step a is taken.

32. A diagnostic kit for confirming the occurrence of a traumatic brain injury event comprising at least three antibodies which are specific for each of three different marker proteins, said antibodies capable of being immobilized on a solid support, wherein
a. a first marker protein is myelin basic protein and a first antibody is specific therefor,
b. a second marker protein is the beta isoform of S100 protein and a second antibody is specific therefor, and
c. a third marker protein is neuronal specific enolase and a third antibody is specific therefor, and
at least three labeled antibodies, each of said labeled antibodies binding to one of said marker proteins, and
e. means for comparing said three markers to specific threshold values of each of the markers to determine the presence of statistically significant concentrations thereof of at least about two standard deviations above normal levels;
wherein said step of comparing said three markers confirms the occurrence of a traumatic brain injury event.

33. A diagnostic kit as defined in claim 32 wherein each of said three antibodies are immobilized on the same solid support.

34. A diagnostic kit as defined in claim 32 wherein at least one of said three antibodies is immobilized on a first solid support and at least another of said three antibodies is immobilized on a second solid support.

35. A diagnostic kit as defined in claim 32 wherein at least one of said labeled antibodies comprises an enzyme-labeled antibody.

36. A diagnostic kit as defined in claim 32 and further including a fourth antibody which is specific for a fourth marker protein, wherein said fourth marker protein is cell type specific with respect to one of said first, second or third markers and has a correspondingly higher molecular weight than said first, second or third marker, and a fourth labeled antibody which binds to said fourth marker protein.

37. A diagnostic kit as defined in claim 36 wherein said fourth labeled antibody comprises an enzyme-labeled antibody.
